(19) United States
(12) Patent Application Publication
(10) Pub. No.: US 2024/0065570 A1
(43) Pub. Date: Feb. 29, 2024

(54) SENSOR AND SYSTEM FOR MONITORING

(71) Applicant: MariCare Oy, Vantaa (FI)

(72) Inventors: Göran SUNDHOLM, Tuusula (FI); Juha LINDSTRÖM, Espoo (FI); Jarmo KIVINEN, Helsinki (FI); Jari EKBLAD, Espoo (FI)

(73) Assignee: MariCare Oy, Vantaa (FI)

(21) Appl. No.: 18/280,107

(22) PCT Filed: Feb. 14, 2022
(86) PCT No.: PCT/FI2022/050089

§ 371 Date: Sep. 1, 2023

(30) Foreign Application Priority Data
Mar. 4, 2021 (FI) 20215240
Jun. 2, 2021 (FI) 20210030

(51) Int. Cl. A61B 5/05; A61B 5/00; A61B 5/08; A61B 5/113

(52) U.S. Cl. CPC A61B 5/05; A61B 5/0826; A61B 5/113; A61B 5/4815; A61B 5/746; A61B 5/447; A61B 2503/08

(57) ABSTRACT

A method, a sensor and a system for the sensor includes a device for processing the measurement signal of the sensor, such as measuring electronics, and a device for communicating measurement results and/or data relating to the measurement results for further processing. The sensor is a radar-based sensor, such as a frequency-modulated continuous-wave MIMO radar-based sensor, configured to detect persons in the monitored area and to measure and detect movement, such as breathing frequency, location, velocity and/or shape of the monitored person. The sensor or a monitoring system connected to the sensor is configured to determine at least one of the following states of the person: breaks or interruptions with breathing of the monitored person, e.g. in order to recognize sleep apnea, and/or immobility of the monitored person, e.g. in order to avoid bedsores or pressure ulcers, and the sensor and/or the monitoring system is configured to provide an alarm based on the determined state of the person.

### FIELD OF THE INVENTION

The invention relates to a method, a sensor and to a system, by means of which persons in a monitored area can be observed, tracked and monitored.

### BACKGROUND OF THE INVENTION

The monitoring of the condition of elderly people in a home environment is indispensable, if it is desired to lengthen the possibility of an aging population coping in their home environment. Safety bracelet systems are nowadays widely used for these kinds of applications. Their weakness is that the user must wear the bracelet continuously and must be able to press the alarm button in an emergency. There are also bracelets that check the state of health of the user, but they have the same problems as described above, and additionally, there are further problems with false alarms.

There have also been presented solutions, in which a film of piezoelectric material is installed on the floor, in which the film registers pressure changes caused by movement on the surface of the floor. Also known in the prior art is the use of sensors to be installed on the floor, or under it, that detects the presence and movements of people, without requiring a change in pressure, but functions by means of capacitive sensors.

The possibility of using video cameras, movement detectors that are based e.g. on detecting infrared light, or e.g. ultrasound sensors, for monitoring the condition and state of elderly people is also presented in prior art. For example, WO2012164169 document discloses a method and a system that are based on ultrasound technology for tracking objects.

Some prior art solutions are known, which use millimeterwave (MMW) radar for tracking persons.

A drawback of observation and monitoring systems, known in the prior art, is that they are not able to provide reliable measurement results in different kinds of circumstances. For example, breathing interruptions or breaks are not sensed reliably in the prior art solutions e.g. to determine a sleep apnea situation and no further actions are implemented based on the recognized situations. The same problems have been noted with other states or conditions of a person, e.g. if a person is immobile for a long time and/or if a person is snoring when he or she is sleeping. In addition to that, observation and monitoring systems of the prior art are complex, and expensive to manufacture, install and maintain. For example, existing radar-based sensors cannot be installed in the optimal orientation in the corner of the ceiling because they lack the means to compensate for the orientation.

### BRIEF DESCRIPTION OF THE INVENTION

The sensor, according to the invention, is a sensor configured to measure the state and/or attitude of a person, and the system, according to the invention, comprises at least one such sensor. In the solution, according to the invention, the sensors can be installed e.g. on a stand, ceiling, floor or wall. The solution, according to the invention, can be used e.g. for monitoring the condition and state of elderly people, for example, in their own homes or in retirement homes. One of the conditions or states of the person are breathing interruptions or breaks, which can be sensed with the sensor or a system connected to the sensor, e.g. in order to determine a sleep apnea situation. One of the conditions or states of the person, which can be detected is immobility of the monitored person, e.g. in order to avoid bedsores or pressure ulcers. One of the conditions or states of the person, which can be detected is snoring of the monitored person. The person and/or others can also be warned with an alarm based on the notifications.

By using the sensor, according to claim 1, the method of claim 14 and the system according to claim 25, the problems of the state of the prior art can be eliminated. The invention is characterized by what is disclosed in the claims.

The invention relates to a sensor for observing the presence, location, movement and/or attitude of a person in a monitored area. The sensor comprises a means for processing the measurement signal of the sensor, such as measuring electronics, and a means for communicating the measurement results and/or data relating to the measurement results for further processing. The sensor is a radar-based sensor, such as a frequency-modulated continuous-wave MIMO radar-based sensor, configured to detect persons in the monitored area and to measure and detect movement, such as breathing frequency, location, velocity and/or shape of the monitored person. The sensor or a monitoring system connected to the sensor is configured to determine at least one of the following states of the person: breaks or interruptions with breathing of the monitored person, e.g. in order to recognize sleep apnea or immobility of the monitored person, e.g. in order to avoid bedsores or pressure ulcers. In one embodiment of the invention the state of the person is snoring of the monitored person. The sensor and/or the monitoring system is configured to provide an alarm based on the determined state in the person's breathing.

In one embodiment of the invention, the alarm is a local alarm comprising an audible alarm, e.g. via a speaker, headphones or a hearing aid device, a visual alarm, such as a light, and/or an alarm, causing vibrations to the bed, mattress and/or to the monitored person, e.g. suffering from breathing interruptions or sleep apnea, immobility and/or snoring. In one embodiment of the invention, the local alarm is an alarm on a wearable device, such as a bracelet or a watch, wherein the alarm is vibrating on the wearable device and/or there is an electric shock caused by the wearable device.

In one embodiment of the invention, the sensor is configured to recognize breaks or interruptions in the person's breathing, e.g. a sleep apnea situation, in such a way that if no movement of the person caused by breathing is determined over a predefined duration, this is recognized as a break or interruption in breathing and/or sleep apnea.

In one embodiment of the invention, the sensor and/or the system is configured to recognize immobility of the person in such a way that if no movement of the person is determined for a predetermined duration, the person is determined to be immobile. The predetermined duration can be set to be a time period, in which risk of bedsores and/or pressure ulcers to the person is low.

In one embodiment of the invention, the sensor and/or the system is configured to provide the local alarm until the person is determined to have moved, woken up and/or to started to breathe again.

In one embodiment of the invention, the sensor or a system is configured to provide a remote alarm based on the determined state of the person, e.g. breaks or interruptions in the person's breathing, immobility of the person or snoring of the person, by sending a message or alarm to a remote system and/or to a mobile device.

In one embodiment of the invention, the remote alarm is provided, if the person does not respond to the local alarm, e.g. if the person does not move, wake up and/or start breathing in response to the local alarm after a predetermined time.

In one embodiment of the invention, the sensor is arranged on a stand, floor, ceiling or wall of a room in a home environment or a hospital environment, e.g. arranged beside or above a bed so that the measurement area of the sensor covers at least part of the bed and/or a person lying on the bed.

In one embodiment of the invention, the sensor comprises a first operating mode and a second operating mode. In the first operating mode the sensor is configured to track movement of the monitored person and in the second operating mode the sensor is configured to measure and/or further analyze measurements relating to a part of the monitored area, in which the movement of a person was observed in the first operating mode in order to observe the status of the person, e.g. interruptions or breaks in breathing of the monitored person or immobility of the person. In one embodiment of the invention the status of the person which can be determined in the second operating mode comprises snoring of the person.

In one embodiment of the invention in the second operating mode, the sensor is configured to analyze the measurement signal in such a way that the phase of the measurement signal is determined in order to observe movement of the object, such as heartbeat and/or breathing and/or immobility and/or snoring.

In one embodiment of the invention, the sensor is a radar sensor configured to observe the elevation, azimuth, movement and/or distance of objects, e.g. with continuous-wave radar technique, such as a frequency-modulated continuous-wave (FMCW).

In one embodiment of the invention, the sensor comprises a means for detecting the orientation of the sensor, such as an accelerometer, and the sensor or the system is configured to take the detected orientation of the sensor into account when determining the presence, location, movement and/or attitude of the monitored person, e.g. by compensating the measurement results based on the detected orientation.

In one embodiment of the invention, the sensor is configured to determine sleep quality of the monitored person. In one embodiment of the invention, the sensor and/or a system is configured to determine sleep quality based at least in part on the movement and/or breathing frequency of the monitored person, e.g. by tracking the combination of the even breathing frequency and the amount of body movement. The determined sleep quality of the monitored person can comprise information related to at least one of the following: how long the person has slept, how much the person has moved while sleeping, what kind of sleep phases the monitored person has had while sleeping, e.g. how much deep sleep the person has had.

The invention relates also to a method for observing the presence, location, movement and/or attitude of a person in a monitored area. A sensor is used in the method, which comprises a means for processing the measurement signal of the sensor, such as measuring electronics, and means for communicating measurement results and/or data relating to the measurement results for further processing, wherein the sensor is a radar-based sensor, such as a frequency-modulated continuous-wave MIMO radar-based sensor. In this method the sensor detects the presence of persons in the monitored area and measures and detects movement, such as breathing frequency, location, velocity and/or shape of the monitored person, and the sensor or a monitoring system connected to the sensor determines at least one of the following states of the person: breaks or interruptions with breathing of the monitored person, e.g. in order to recognize sleep apnea, immobility of the monitored person, e.g. in order to avoid bedsores or pressure ulcers. In one embodiment of the sensor or a monitoring system connected to the sensor determines at least one of the following states of the person: snoring of the monitored person. The sensor and/or a monitoring system provides an alarm, e.g. local alarm, based on the determined state of the person.

In one embodiment of the invention, the local alarm is an audible alarm, e.g. via a speaker, headphones or a hearing aid device, a visual alarm, such as a light, and/or an alarm causing vibrations to the bed, mattress and/or to the monitored person, e.g. the monitored person suffering from breathing interruptions or sleep apnea, immobility and/or snoring.

In one embodiment of the invention, the local alarm is an alarm on a wearable device, such as a bracelet or a watch, wherein the alarm is vibrating on the wearable device and/or there is an electric shock caused by the wearable device.

In one embodiment of the invention, the sensor recognizes breaks or interruptions in the person's breathing, e.g. a sleep apnea situation, in such a way that if no movement of the person caused by breathing is determined over a predefined duration, this is recognized as a break or interruption with breathing and/or sleep apnea.

In one embodiment of the invention, the sensor and/or the system recognizes immobility of the person in such a way that if movement of the person or movement above a threshold level is not determined for a predetermined duration, the person is determined to be immobile. The predetermined duration can be set to be a time period in which the risk of bedsores and/or pressure ulcers to the person is low.

In one embodiment of the invention, the local alarm is provided until the person is moving, waking up and/or starts to breathe again.

In one embodiment of the invention, the sensor or a system provides a remote alarm based on determined state of the person, e.g. breaks or interruptions in the person's breathing, immobility of the person or snoring of the person by sending a message or alarm to a remote system and/or to a mobile device.

In one embodiment of the invention, the remote alarm is provided, if the person does not respond to the local alarm, e.g. if the person does not move, wake up and/or start breathing in response to the local alarm after a predetermined time.

In one embodiment of the invention, the sensor comprises a first operating mode and a second operating mode. In the first operating mode the sensor tracks movement of the monitored person and in the second operating mode the sensor measures and/or further analyzes measurements relating to a part of the monitored area, in which movement of a person was observed in the first operating mode in order to observe the status of the person, such as interruptions or breaks in breathing of the monitored person, immobility of the monitored person and/or snoring of the person.

In one embodiment of the invention, in the second operating mode the sensor analyzes the measurement signal in such a way that the phase of the measurement signal is determined in order to observe the movement of the object, such as heartbeat and/or breathing, snoring or immobility.

In one embodiment of the invention, the sensor comprises a means for detecting the orientation of the sensor, such as an accelerometer, and the sensor or the system takes the detected orientation of the sensor into account, when determining the presence, location, movement and/or attitude of the monitored person, e.g. by compensating for the measurement results based on the detected orientation.

In one embodiment of the invention, the sensor determines sleep quality of the monitored person. In one embodiment of the invention, sleep quality can be determined based at least in part on the movement and/or breathing frequency of the monitored person, e.g. by tracking the combination of even breathing frequency and the amount of body movement. The determined sleep quality of the monitored person can comprise information related to at least one of the following: how long the person has slept, how much the person has moved while sleeping, what kind of sleep phases the monitored person has had while sleeping, e.g. how much deep sleep the person has had.

The invention relates also to a system for observing the presence, location, movement and/or attitude of one or more objects in a monitored area. The system comprises at least one sensor according to the invention. The sensor or sensors are fitted in the monitored area, e.g. on a stand, a floor, wall and/or ceiling.

In one embodiment of the invention, the system is configured to provide the local alarm and/or the remote alarm.

In one embodiment of the invention, the sensor is arranged beside or above a bed so that the measurement area of the sensor covers at least a part of the bed and/or a person lying on the bed, and the sensor is arranged to measure a person on the bed, e.g. in a home environment or hospital environment.

One advantage, among others, is that the system is able to reliably observe movement of the person and, in addition, also health related functions of the person such as heartbeat, breathing, immobility and/or snoring with a single integrated sensor. The system is also able to provide a local alarm in a situation in which the attention of the person is needed, e.g. in a breathing interruption situation, e.g. caused by sleep apnea, immobility of the person, or when the person is snoring, so that the person can move wake up and e.g. start to breathe again. No sensors attached to the user or person are needed and the sensor can monitor the person in a wireless manner, e.g. in a home environment or hospital environment.

Also, in one embodiment, a system with multiple sensors can be provided so that a bigger area, e.g. areas outside the bed areas, can be monitored. An advantage of one embodiment of the invention, in which multiple radar-based sensors are used in monitoring the persons, the sensors and their operation can be arranged so that they do not cause interference to each other despite the sensors are at least partly operating in the same space.

With the above-described solution of the invention, a monitoring system is provided which is known to be able to provide reliable measurement results in different kind of circumstances and is easy install and maintain.

### BRIEF DESCRIPTION OF THE FIGURES

The invention is illustrated with the following drawings, of which:

FIG. 1 presents the components of one example embodiment of the system of the invention, in the area to be monitored,

FIG. 2 presents the operation of one example embodiment of the system of the invention,

FIG. 3 presents an example embodiment of a sensor, according to the solution of the invention,

FIGS. 4A-B present one embodiment of the invention, in which sensors are arranged to a room, e.g. in a hospital environment, and

FIGS. 5A-B present one embodiment of the invention, in which sensors are arranged in a room, e.g. in a home environment,

FIG. 6 presents one example embodiment of the system, according to the invention, and

FIG. 7 presents an example of a processing pipeline of the system, according to one embodiment of the invention.

### DETAILED DESCRIPTION OF THE INVENTION

In the solution of the invention the sensors are radar-based sensors, which can detect presence and movement of an object. The monitored object can be e.g. an elderly person or some other person benefitting from supervision. The sensor can be installed on a stand, on a surface, e.g. on a wall, door, floor or ceiling, and/or in the proximity of a surface, such as e.g. floor surfaces, wall surfaces, door surfaces or ceiling surfaces of an apartment, room and/or of the area to be monitored to which the object has access. The sensor can also be used to observe vital functions of the monitored person, such as the breathing, e.g. breathing frequency, and even the heart beating rate of the person. This feature allows monitoring of, for example, a sleeping or person, who has fallen and gives an alarm if any unusual phenomenon are observed.

In the solution according to the invention, the system comprises at least one sensor and can further comprise measuring electronics producing sensor observations by means of the sensors, and a processor configured to process the sensor observations, and/or a central unit comprising a memory, which the central unit is e.g. a data processing device. For the purposes of this function, the central unit of the system can comprise the necessary software and information about the characteristic properties of the signals being detected. In general, the measuring electronics and/or the central unit can deduce information from a signal received via a sensor. The system can have a central unit, which can manage one or more sensors or sensor groups. In one embodiment of the invention, one sensor group comprises e.g. the sensors in the same space, such as in the same room.

An area to be monitored with sensors can be the whole area or only a part of some area. The area to be monitored can comprise e.g. of one or more rooms and certain parts of the area, e.g. fixed installations such as cupboards, can be left outside the area to be monitored. In one embodiment, in which a sleeping person is monitored, the sensor can be arranged in connection with the bed, above the bed and/or beside the bed so that the monitoring area of the sensor covers at least a part of the bed or a person lying on the bed.

In the solution according to the invention, the sensor detects persons in the monitored area and measures and detects the location, velocity and/or shape of the monitored person. In one embodiment of the invention the sensor is configured to observe the object based on signal strength and/or by filtering out probable false measurement results.

The sensor can be installed on a stand, on a surface, e.g. on a wall, door, floor or ceiling, and/or in the proximity of a surface, such as e.g. floor surfaces, wall surfaces, door surfaces or ceiling surfaces of an apartment and/or of the area to be monitored to which the object has access. In one embodiment of the invention the sensor or sensors are installed in the corner of the space to be monitored right below the ceiling tilted towards the centre of the space. A typical tilt angle can be e.g. 15 degrees. This gives the sensor a good view over obstacles such as furniture. In one embodiment of the invention the sensor or sensors are installed on a wall or in a corner of the space to be monitored, typically above the floor-level plane, e.g. at a height of approx. 40-150 cm from the floor. The field of view of the sensor can be e.g. approx. 90 degrees on the horizontal plane.

The sensor can comprise e.g. a millimeterwave (MMW) radar, which can operate for example with the MIMO radar principle. In one example embodiment, there can be, for example, three transmitter and four receiver antennas. In that example this forms a virtual antenna of 12 elements. With the sensor of the invention, it is possible to observe the elevation, azimuth, movement and distance of objects with good accuracy. E.g. FMCW (frequency modulated continuous wave) technique can be used for the radar.

The sensor can measure and detect movement, such as breathing frequency, location, velocity and/or shape of the monitored person. The sensor is configured to determine status of the person, such as breaks or interruptions with breathing of the monitored person, e.g. in order to recognize sleep apnea and/or immobility of the person. In one embodiment of the invention the determined status of the person comprises person's snoring. The sensor and/or a monitoring system is configured to provide an alarm, such as a local alarm, based on the determined condition of the person's breathing.

The system of the invention can be used to detect high risk of pressure ulcers. Pressure ulcers (PUs; i.e., bed sores, pressure sores) are localized areas of tissue damaged as a result of excessive pressure and shearing forces. Pressure ulcers occur most often in individuals, who have limited mobility and are exposed to prolonged periods of pressure, for example, while lying motionless in the bed. There are also statistically significant increases in prevalence in older age groups. Most PUs can be avoided with effective risk assessment and subsequent targeted interventions.

In a nursing home, PUs are significant problems for residents with limited mobility. To avoid PUs, the nurses must visit the sleeping residents at regular intervals to change their positions e.g. from one side to the other. Obviously, this not only increases the workload of the already busy nurses but also disturbs the sleep of the other residents, even if they have already been changing positions themselves. Also, sometimes it might be difficult to assess the risk of PUs for some residents. They might have good mobility but still sleep too motionless.

This invention can solve these problems with a system that is capable of tracking the location and/or position of a resident, monitoring his movements, while resting and warning the care givers, if the resident is at risk of getting PUs due to insufficient motion. The purpose of the location tracking is to allow the system to separate different sources of movements e.g. a nurse visiting the area. It also allows the system to dynamically adapt to different locations of the residents being monitored.

In one embodiment of the invention, the sensor and/or the system recognizes immobility of the person in such a way that if no movement of the person is determined for a predetermined duration, the person is determined to be immobile. In this way the risk of pressure ulcers for the monitored person can be kept low. The predetermined duration can be set to be a time period, in which the risk of pressure ulcers to the person is low. In one embodiment, if the person does not respond to a local alarm caused by immobility, another person, e.g. nurse, can be warned to move the person.

In one embodiment of the invention, the system uses an FMCW radar operating in the millimeter wave band with an antenna array to track the precise location of the person. The system includes a user interface or another configuration interface that can be used to specify the locations of beds, couches and other locations of interest and to save these in the room configuration data store. A CPU receives the location of the person from the radar and consults with the room configuration to determine if the resident is located in a bed or another place for resting. When this happens, the CPU tells the radar to focus on the location of interest and start monitoring the fine motions. This focusing can be done e.g. by using beam forming with the antenna array to amplify the signal originating from the direction of interest.

Large motions can be detected by observing changes in the range or the Doppler spectrum of the signal. Small motions can be detected by observing changes in the phase angle of the signal. By combining these methods and applying appropriate thresholding, the system can detect movements that are large enough to e.g. prevent PUs. In one embodiment of the invention, the system includes a timer that is reset in the beginning, and whenever a large enough motion is detected (the motion is over a set threshold). In one embodiment of the invention, the system can comprise a user interface, which illustrates the length of duration a resident has been motionless. If the duration gets too long, the system triggers an alarm to notify the nurses about the risk of PUs. The alarm can be delivered to a mobile phone or any other alarm channel used by the nurses.

In one embodiment of the invention, the sensor and/or the system recognizes snoring of the person. The snoring can be recognized in one embodiment only by a microphone. In one embodiment, snoring can be determined based on measurements of the radar-based sensor. In one embodiment, a combination of measurements from a radar-based sensor and a microphone can be utilized. In this way the person can be warned with a local alarm, if he or she is snoring. In one embodiment of the invention utilizing the microphone, the microphone can be integrated and/or connected to the sensor.

In one embodiment of the invention, the sensor is configured to determine sleep quality of the monitored person. The determined sleep quality of the monitored person can comprise information related to at least one of the following: how long the person has slept, how much the person has moved while sleeping, what kind of sleep phases the monitored person has had while sleeping, e.g. how much deep sleep the person has had. In one embodiment of the invention, sleep quality can be determined based at least in part on the movement and/or breathing frequency, e.g. even breathing frequency, of the monitored person sensed by the sensor. In one embodiment of the invention, determination of sleep quality can be implemented at least in part by tracking the combination of even breathing frequency and the amount of body movement.

In one embodiment of the invention, the sensor is configured to track the movement of the observed person in the first operating mode by analysing the signal reflected from the person, e.g. Doppler frequency, the range and angle of arrival of the signal. In one embodiment of the invention, the sensor is configured to track heartbeat and/or breathing of the monitored person in the second operating mode by analyzing the phase of the measurement signal. In one embodiment of the invention, wherein the sweep time of the sensor is longer in the second operating mode than in the first operating mode. In one embodiment of the invention the second operating mode can differ from the first operating mode only by the digital signal processing algorithm applied to the signal.

In one embodiment of the invention, in the second operating mode, tracking of the persons is not performed. In one embodiment of the invention one sensor can use the first and second operating mode at the same time, e.g. so that the first operating mode is always used and the second operating mode is activated when it is needed, and when it is not needed, the second operating mode is deactivated. In one embodiment of the invention, one sensor can use the first and the second operating mode in an interleaved manner.

The sensor can be configured to activate the second operating mode based on detecting that the monitored person is not moving, has fallen and/or the speed of the monitored person is slower than a predefined threshold value. The sensor can be configured to deactivate the second operating mode based on detecting that the monitored person is not determined as fallen, the person is moving and/or the speed of the monitored person is higher than a predefined threshold value.

In the second operating mode, the sensor can be configured to analyze the measurement signal in such a way that the phase of the measurement signal is determined in order to observe the movement of the person, such as heartbeat and/or breathing. In one embodiment of the invention, in the second operating mode, the sensor and/or the measuring electronics of the sensor are configured to analyze the measurement signal from the area and/or certain distance around the area relating to the determined azimuth, elevation and/or distance from the sensor of the person determined in the first operating mode.

In one embodiment, the system comprises at least two sensors and is configured to detect and measure the persons in the monitored area based on the measurement signal of at least two sensors, which can monitor the same area and/or a different part of the monitored area. For example, the measurement area of the sensors can overlap for example at a certain part of the area.

In embodiment of the invention, in an apartment or a nursing home, there can be at least one radar in each room. In this case, the radars would be interfering with each other if no corrective measures are taken. In one embodiment, a division of modes and/or several radars to specific time slots is presented, so that several radars can be used simultaneously close to each other without causing interference. The transmissions of the sensors can for example be synchronized and carried out in interleaved manner in such a way that the sensors are able to observe the same person and/or the same room.

In one embodiment of the invention, different sensors can be in different operating modes, e.g. some sensors determine a stationary object while the second operating mode is activated, while the other sensors use only the first operating mode to monitor movement of the objects and to search for stationary objects.

The system can send a notification of a fall, if a person is interpreted as having fallen and/or if the vital functions of the monitored person, such as tracked heartbeat and/or the monitored person's breathing, is not within the predefined limits. In one embodiment, a notification and/or a fall notification comprises sending an alarm or message to a person and/or an organization, monitoring the health of the person, e.g. as a message to a phone, as an alarm and/or e.g. to a nurse, to relatives or to an emergency centre.

In one embodiment of the invention, fixed objects, such as beds or sofas, where the person can lay down, can be determined by the user with the sensor and/or a sensor system, and the sensor does not determine the person as fallen in the areas of these fixed objects.

In one embodiment of the invention, the sensor can distinguish objects from the observed persons by the determined elevation of the observed object, e.g. in such a way that when the elevation of the determined objects is essentially constantly under a certain threshold elevation value, the object can be recognized as not being a person.

In some applications, it is advantageous to first chart the unchanged area, i.e. to chart the measuring information of the sensors when mainly stationary and unmovable objects and structures are in place. This type of situation is e.g. in a residential apartment when the furniture is in position but there are no people, pets or robots in the apartment. This charted information can be recorded in the system, e.g. in a memory that is located in the central unit or in a memory means that is in connection via a data network, which memory means can be e.g. in a control centre or service centre. For this purpose, a memory means can be integrated into the arrangement, so a memory means can be in the central unit or connected to it via a data network.

According to one embodiment of the invention, the system charts the unchanged area continuously or at defined intervals, in which case the system is able to detect e.g. changes in the area caused by new furniture or by changes in the location of furniture. In this way the system is able to adapt gradually to changes occurring in the area to be monitored.

In one embodiment of the invention, the sensor or system is configured to detect a person falling and/or sitting by the determined elevation of the person, e.g. such that when the elevation of the person is under certain threshold elevation values, the person can be determined to have fallen. In one embodiment of the invention, the elevation of a person is tracked and filtered with a filter, such as a Kalman filter or a low pass filter, in order to prevent false alarms due to noisy measurements.

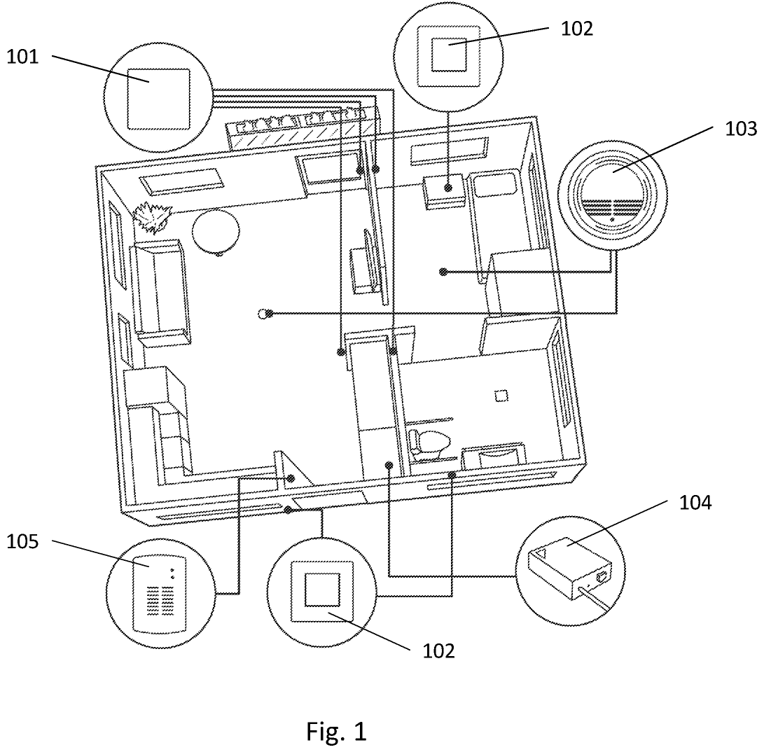

FIG. 1 presents the components of an embodiment of the system, according to the invention in the area to be monitored. The sensor 101 or sensors to be used in the invention are arranged in connection with the area to be monitored in such a way that by means of the sensor 101 or sensors, the area to be monitored can be monitored. If sensors to be installed on top of a surface, e.g. a wall, floor or ceiling surface are used, they can be fastened to the surface e.g. with double-sided tape or with a sticker strip, in which case they can easily be removed. The sensors 101 can be connected wirelessly or by wireline to the gateway 104, which collects measured values obtained from the sensors 101 or status information formed by the sensors 101, e.g. the objects detected, the state of health of the objects, such as persons, and/or the movement and attitudes of the objects. The gateway 104 sends the information onwards e.g. to a control centre or to another body that supervises the area and/or the objects, such as persons, therein. The transfer of information between the system and some recipient can be performed e.g. using a phone connection, a wireline broadband connection or wireless connections. It is advantageous in the data transfer to take into account issues relating to data security and privacy, which many official regulations also address.

In one embodiment of the invention, the sensor 101 or sensors comprise their own central unit and the central unit of a sensor is in connection with the gateway 104. In a second embodiment of the invention, the central units of the sensor 101 or sensors are integrated into a gateway 104.

It is possible that some of the functions of the central unit or of the gateway 104 are performed elsewhere via a data network connection, e.g. in a central control room or service centre.

According to one embodiment of the invention, an alarm signal can be given by the system in the space being monitored, which lasts a predetermined period of time. This alarm signal can be given as a local alarm, e.g. before the sending of an alarm or notification, and it can be given via a light alarm unit and/or a sound alarm unit of the system. The light alarm unit and/or sound alarm units can be in each different part, e.g. a room of the premises. This functionality can also be integrated into the sensors, e.g. into all the sensors or only some of the sensors.

The system, according to the invention, can also comprise a call pushbutton 102. After pressing the call pushbutton, the system can connect to e.g. nursing personnel, security personnel, or it can perform various alarm procedures. The call pushbutton can be wireless and it can be adapted to function without batteries.

The notification procedures and alarm procedures, according to the system of the invention, can include e.g. activating a local alarm, indication signalling (such as a buzzer, light, siren, alarm clock, etc.), making contact with an alarm centre or service centre, a care provider or a relative. In some cases, an alarm can also be sent directly to the person being monitored or to the user, e.g. by means of speech synthesis or a speech recording. For performing these tasks, the arrangement can comprise means needed for processing time data, such as e.g. a clock circuit.

The system, according to the invention, can also comprise fire detectors 103, which can be in connection with another system via a wireline or wireless connection. If the fire detectors 103 warn of a fire, alarm procedures can be performed, e.g. by sending an alarm message to a control centre or to the rescue authorities.

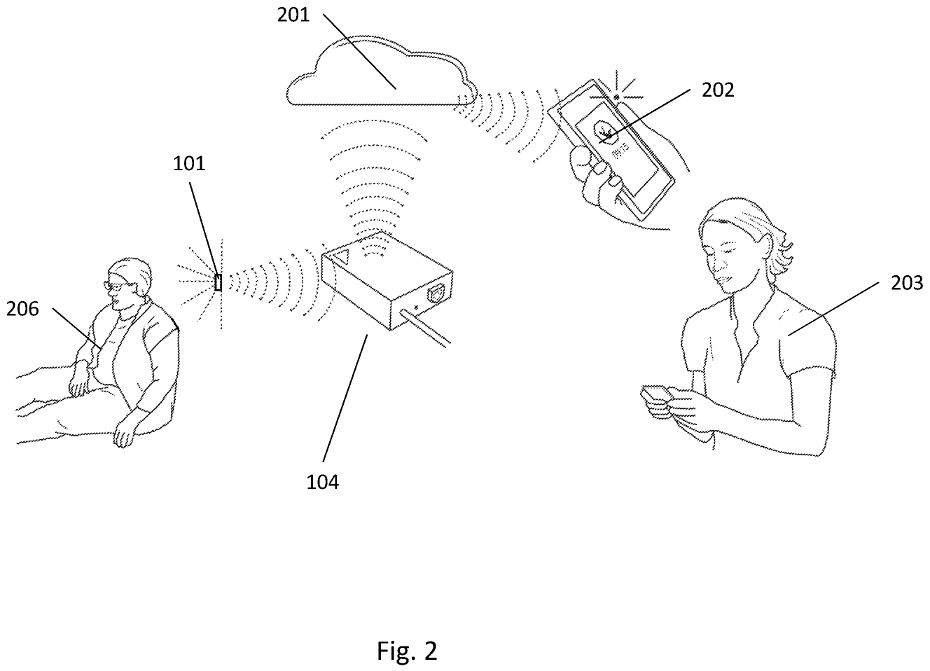

FIG. 2 presents the operation of an embodiment of the system, according to the invention, in which the state of health or attitude of a person 206 in the area being monitored is monitored.

If the sensors 101 of the system detects that an object, such as a person, 206 in the area being monitored has fallen and/or the vital functions, such as breathing frequency, of the person are not at the acceptable level, the system can send a notification.

In one embodiment of the invention, the system examines the information measured by a number of sensors, e.g. by all the sensors in the area being monitored, and a notification, e.g. a remote alarm, is only sent if no other persons are detected in the area by the sensors.

In the situation in the embodiment presented in FIG. 2, in which the system sends a message e.g. because of the falling of a person or because of determined vital functions of the person, the sensor 101 sends the information about the situation to the gateway 104 of the system and the gateway 104 sends the information and/or an alarm onwards to the server 201 e.g. via an Internet connection or via some other connection. From the server 201, the information and/or alarm is sent to a body monitoring the health of the person, e.g. as a message to a mobile phone 202, as an alarm and/or e.g. to a nurse 203, to relatives or to an emergency centre. In this way, e.g., information about the falling or health of the person reaches the necessary people or organizations and the person who fell receives help as quickly as possible. In one embodiment of the invention, the system can send information directly from the gateway 104 to an organization or a person monitoring the health of the monitored person.

The processor, central unit and/or measuring electronics, used in the solution of the invention can be integrated into the sensors or they can be disposed separately or in separate units. In an embodiment of the invention, with a software executed by the processor, the sensor or system can interpret the movements observed with at least one sensor and can give an alarm, if the alarm conditions defined for the program are fulfilled.

In one embodiment of the invention, only some of the sensors of the area to be monitored have the functionality, enabling the issuing of an alarm signal as described above. For example, the sensors in only some rooms, such as in the living room, can be provided with this functionality and the sensors in other rooms send a notification onwards immediately after a fall is detected and/or measurement results of a monitored person are not in an acceptable and/or in a predefined range. In one embodiment of the invention, only some of the sensors in one space, such as in a room, comprise the functionality, enabling the issuing of an alarm signal as described above.

The system can also comprise a control centre and the predetermined information concerning the presence, location, movement and/or attitude of the object can be sent to the control centre. The alarm terms used by the system can be changed, e.g. on the basis of presence information, which can be e.g. received from an RFID reader. A notification can be sent or an alarm can be given e.g. to an external alarm system or wirelessly to a central server of the system, from which server the alarm is directed onwards.

The system can also have a memory means, in which the system is adapted to record a measurement signal, or information derived from it, for observing the chronological dependency of the area being monitored and the behaviour of people. By means of this, the system can give an alarm e.g. if a person being monitored has not got out of bed or visited the kitchen for a certain time, or if the person has gone to the toilet too often or if the vital functions of the observed person, such as breathing or heartbeat, have changed during a specific time. The memory means also enables learning of a more common daily rhythm and the detection of aberrations occurring in it.

In the following, one example embodiment is described. In this example embodiment, first operating mode of the sensor is used to track the presence and movements of people, e.g. in a single room. In this embodiment the tracking is carried out with the measured point cloud data. Doppler range needed is given by

$${f}_{Dmax} = \frac{2 v}{λ}$$

In one example, if the person is moving with a speed of 1 m/s, the needed Doppler range is +−400 Hz and the maximum measurement interval is 2.5 ms at 60 GHz frequency. Inbreathing lasts about 2 seconds. If the corresponding movement is 5 mm, the Doppler range needed is +−1Hz and the sweeping time is one second.

When the system observes that the person has stopped, it can activate the second operating mode, in which it is able to track vital functions of the person, such as heartbeat and/or breathing, such as breathing interruptions and/or frequency. In one embodiment of the invention the system needs to carry out measurements for a certain duration before it can detect the breathing period of a person.

After the vital functions of the persons are determined, the system can deactivate the second operating mode. In one example embodiment, the system can determine the vital functions of the same person periodically, e.g. as long as the person stays stationary. If the system observes stationary persons, it's starts to determine vital functions of these persons by using the second operating mode.

In one example embodiment of the invention, operation in the second operating mode can be implemented, for example, so that when the stationary object has been detected, the point cloud data around an area of the detected object is saved and analyzed. The saved packages can be generated periodically, e.g. every 600 ms. In one embodiment of the invention, the data can be transferred to central control units for analysis. With the analysis of the signal, i.e. the point cloud data, information about small movements of the object can be observed and thus the system is able to determine e.g. breathing activity and/or heartbeat of the person.

In one embodiment of the invention, the sweep time of the sensor is longer in the second operating mode, and because of this, a better signal to noise ratio can be achieved. Also, more TX-antennas can be utilized because there is more time available for measurement. In this way, the angle resolution can be improved. For improving the distance resolution, the frequency sweep range can be increased.

The Doppler frequency can be determined e.g. with Fast Fourier Transformation (FFT). The vital function activity, e.g. heartbeat and breathing activity, can be determined based on the determined Doppler frequency. In one embodiment of the invention, there are more TX antennas used in the second operating mode to increase the spatial resolution. Signal processing can be done for a smaller area because the monitored person is not moving.

In one embodiment of the invention, the sensor and/or the system can comprise a radio-based identification means for identifying a person. The radio-based identification means can be, for example, Bluetooth, Bluetooth low energy (BLE) or Zigbee based means. In this embodiment, the system can recognize the person and a radio-based device carried by the person, such as a bracelet, a watch, a mobile device, a tag, and the measurement results can be linked to the specific recognized person. In this way the system is able to know, who is present in the monitored area and to whom the monitored results relate.

In one embodiment of the invention, the radio-based identification means can comprise an antenna array that makes it possible to more accurately associate the identification devices to their carriers when there are more than one person and device present.

In one embodiment of the invention, the alarms can be automatically disabled, if the identification means detect a certain person such as a nurse in the monitored area.

In one embodiment of the invention, the alarm conditions of the system can include the identity of the person. For example, an alarm can be triggered when an unauthorized person enters certain locations.

In one embodiment of the invention, the radio-based identification means, for example Bluetooth, Bluetooth low energy (BLE) or Zigbee based means, can be used in locating a person or assist in locating the person. The sensor can include several antennas for radio-based identification means, e.g. Bluetooth, BLE or Zigbee antennas to enable direction finding techniques, for example Zigbee, Bluetooth or Bluetooth low energy (BLE) direction finding techniques, e.g. according to Bluetooth 5.1 specification. In one embodiment of the invention, if the radar of the sensor detects movement but the radio-based identification means do not detect a remotely readable tag or device, such as a Bluetooth, BLE or Zigbee tag or device, then the person detected by the radar can be considered a visitor. If, on the other hand, the radar detects a remotely readable tag or device, such as a Bluetooth, BLE or Zigbee tag or device, then the detected person can be identified, and actions can be taken based on the identified person. In one example embodiment, when a resident is in a room and there is also an assisting person, the status of the person or the room can be set in the system to “an assisting person present in the room”. In the same way, an alarm made by a resident can also be acknowledged as the system recognizes that a person, who is not a resident in the room, enters the room. In this case, the alarm can be acknowledged automatically. In one embodiment an alarm is not acknowledged automatically but requires an active identifiable event, e.g. from the user device.

In one embodiment of the invention, identification of the detected person can be done by other means, for example, with surveillance cameras, e.g. arranged in the corridors. In this case, the radar-based sensor detects that someone is entering the room and the system can check information from the surveillance cameras, e.g. from a certain point in time from the surveillance recording, in which a person can be seen to enter the room. In one embodiment, this recording could be linked to the room as an entry event and the entrant could be identified later, if necessary, by looking at the recording. In that case, the identification can be automatic but automatic identification does not have to be implemented if it is not preferred. If automatic identification from the video is used, it can be implemented e.g. based on facial recognition techniques. In one embodiment, if a user can be identified in other ways, facial recognition or video-based recognition is not used. In one embodiment, video-based identification is only used if a person cannot be identified in any other way.

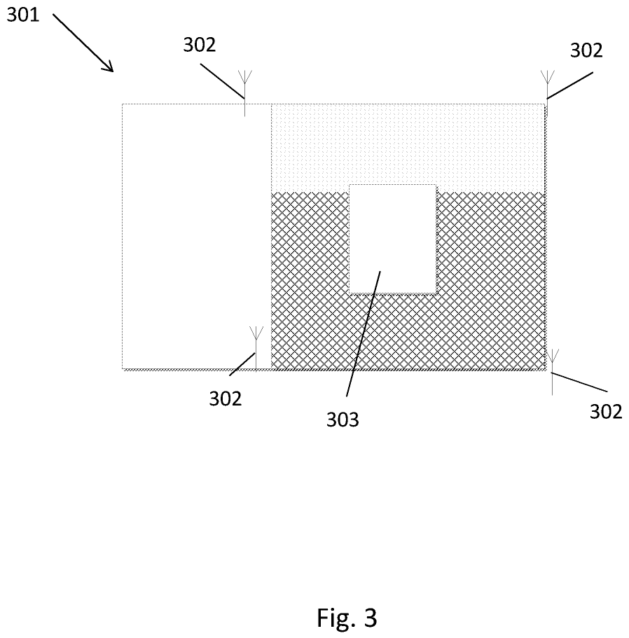

In one embodiment of the invention, in which the radio-based identification means are used, the necessary electronics and antennas can be integrated with the sensor. An example of embodiment is presented in FIG. 3, in which a Bluetooth antenna array is integrated with the sensor 301. The Bluetooth antenna array of FIG. 3 comprises four antennas 302, and the required electronics that control the operation of the identification means and the antennas. The antenna array can be utilized in measuring and detecting Bluetooth devices and tags and e.g. to locate a person carrying a Bluetooth device such as a bracelet, using Bluetooth 5.1 direction finding technique. In one embodiment, the data measured with Bluetooth antenna array is combined, e.g. by the sensor, with the data measured by the radar to increase the location and positioning accuracy of the radar sensor. The antenna or antenna array of the radar 303 (Where is 303 on FIG. 3?) arranged in this embodiment in the centre of the sensors and inside the area formed by the four Bluetooth antennas 302.

In one embodiment of the invention, the sensor according to the invention can be used e.g. in hospital rooms or in rooms where the people are sleeping, and their monitoring is needed. In this embodiment, the sensor can be arranged so that it is able to measure and sense a person who is present in a bed. The sensors can be arranged in the room or in connection with the room so that the monitored area of one sensor covers at least part of one bed. In one embodiment of the invention, the sensors are arranged on the ceiling of the room, e.g. above each bed, for example, one sensor above each bed. In one embodiment of the invention, the sensors are arranged on the wall of the room, e.g. beside each bed, for example one sensor beside each bed. With these embodiments the sensor is able to measure and/or sense the presence of the person in a bed but also vital functions, such as movement, heartbeat and breathing, of the person. One of the advantages of these embodiments is that a sleeping person can be monitored without disturbing him, which is not possible for example with wired sensors. Also monitoring of people, who should be sleeping is easy for the personnel and nurses e.g. in hospital environments with this embodiment. With these embodiments the sensor does not have to comprise its means for detecting the orientation of the sensor.

In one embodiment of the invention. at least one additional sensor, according to the invention, can be arranged in the monitored room or area where people are sleeping. This additional sensor is able to sense and monitor persons that have left their bed. In this case the measurement area of the additional sensor can be bigger than the measurement area of the sensors monitoring beds. The measurement area can cover essentially the whole room, e.g. with single or multiple additional sensors. Also, this additional sensor can be arranged in the room or in connection with the room so that the measurement area of the sensor or sensors cover the room and especially areas outside the beds. In one embodiment, the additional sensor can be arranged on the ceiling, wall and/or corner of the room or on a stand. With this embodiment, the room can be better monitored by the personnel and e.g. an alarm can be given, if people are leaving their beds and/or disturbing other people, who are trying to sleep. These additional sensors also make it possible to monitor people who have left their beds, and e.g. to generate an alarm, if a person falls and/or or if the determined vital functions are not at the predefined and/or acceptable level. With these embodiments, the additional sensor does not have to comprise a means for detecting the orientation of the sensor.

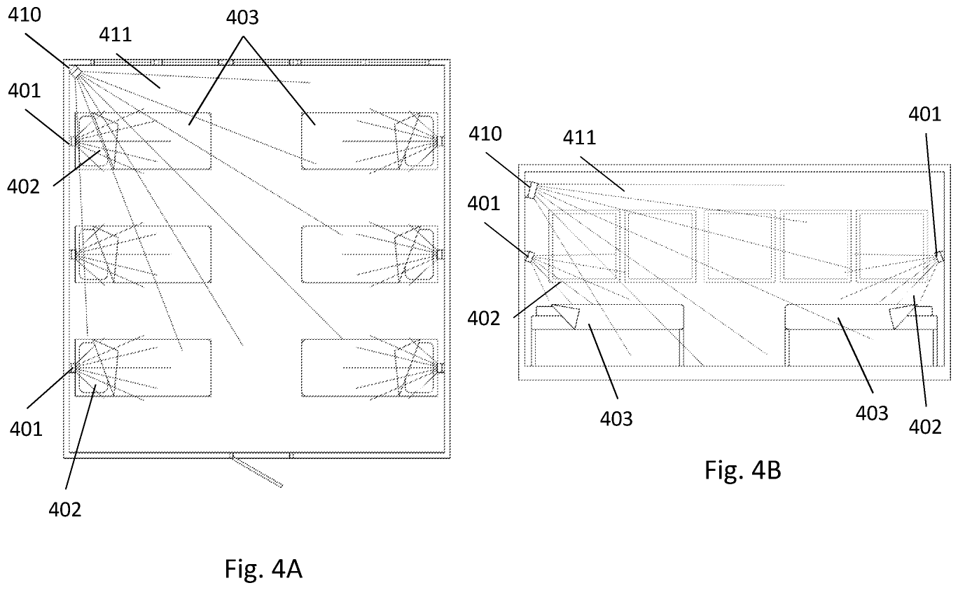

FIGS. 4A and 4B present, as an example, how this embodiment can be implemented in a room, e.g. a hospital room. FIG. 4A presents a room from above and FIG. 4B the same room as a side view. In this example, the embodiment beds 403 are arranged in the room. The Sensors 401 are arranged in the room so that the measurement area 402 of one sensor 401 covers at least part of one bed 403. In one embodiment of the invention, the sensors 401 are arranged on the wall of the room on a stand, e.g. beside each bed 403, for example, there is one sensor beside each bed as presented in FIG. 4A and 4B. An additional sensor 410 is also arranged in the monitored room or monitored area, where people are sleeping. This additional sensor 410 is able to sense and measure the persons that have left their beds 403. In this case, the measurement area 411 of the additional sensor 410 is bigger than the measurement area of the sensors 401 monitoring the beds 403. The measurement area 411 of the additional sensor 410 can cover essentially the whole room. The additional sensor 411 can be arranged on the ceiling, wall and/or corner of the room.

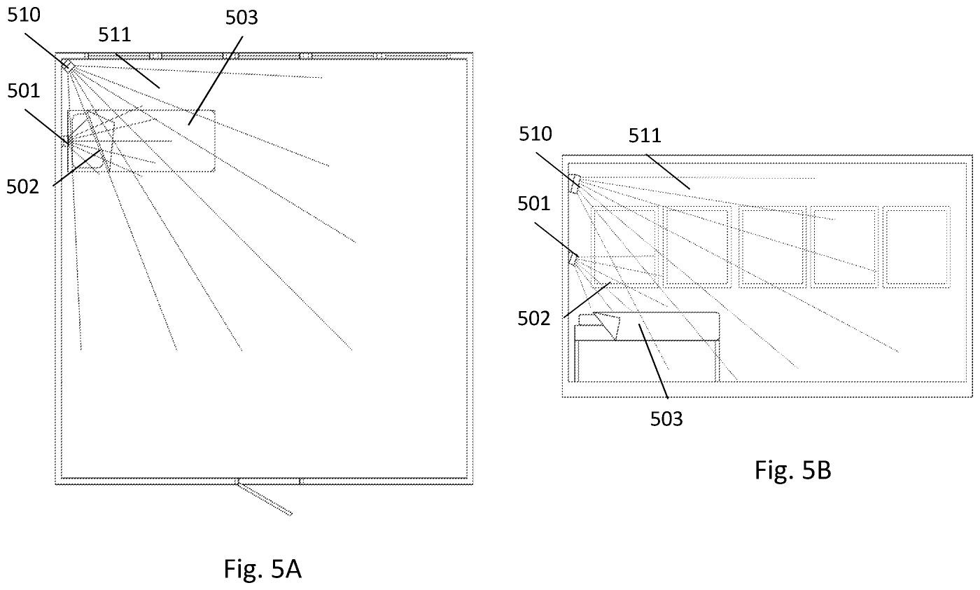

FIGS. 5A and 5B present. as an example. how this embodiment can be implemented in a room, e.g. in home environment. FIG. 5A presents a room from above and FIG. 5B the same room as a side view. In this example embodiment, one bed 503 is arranged in the room. The sensor 501 is arranged in the room so that the measurement area 502 of the sensor 501 covers at least part of one bed 503. The sensor can be arranged on the ceiling, wall and/or corner of the room or on a stand. In one embodiment of the invention, the sensor 501 are arranged on the wall of the room, e.g. beside the bed 503. In one embodiment of the invention, the sensor can be arranged on a ceiling of the room, e.g. above the bed. An additional sensor 510 can also be arranged in the monitored area. This additional sensor 510 is able to sense, measure and monitor the person who has left his/her bed 503. In this case, the measurement area 511 of the additional sensor 510 can be bigger than the measurement area of the sensors 501 monitoring the bed 503. The measurement area 511 of the additional sensor 510 can cover essentially a whole room. The additional sensor 411 can be arranged on the ceiling, wall and/or corner of the room.

In one embodiment of the invention one sensor per room is enough for being able to measure a person in the room in different circumstances. In one embodiment of the invention same sensor can follow fast movement of the person and also monitor the person in bed as the sensor can use first operating mode and second operating mode for different activities of the person. The sensor can monitor the whole room in first operating mode and detect the person's breathing in bed in the second operating mode, e.g. if the sensor is installed close to bed. In one embodiment of the invention, a separate sensor for the bed is thus not needed.

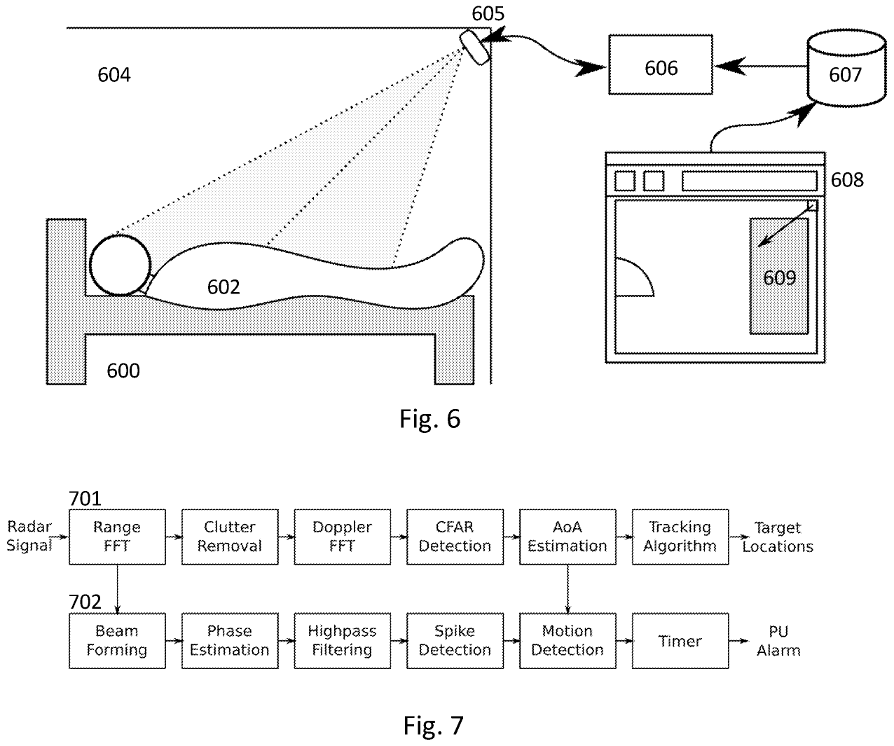

FIG. 6 illustrates at least a part of the components of one embodiment of the system, which can be used to detect health related status of the person, such as pressure ulcers. The FMCW radar 605 is configured to monitor the room 104 and track the people in it. When the person 602 enters the bed 600 the CPU 606 instructs the radar to focus the beam 603 in the direction of the person and resets the motion detection timer. The CPU 606 uses the room configuration data store 607 to determine when the person is in bed. The configuration is entered into the data store 607 with the user interface 608 that allows specifying the location of the bed 609.

The CPU 606 and the room configuration store 607 can be integrated with or within the radar 605 or they can be located in a separate computer. The user interface can be a computer program or a web-based application used with a web browser, accessing the configuration store remotely. The CPU 606 can be a single CPU or it can comprise multiple CPUs, each running their own task of the data processing pipeline.

FIG. 7 illustrates one embodiment of the data processing pipeline used by the system of the invention. The basic radar data processing pipeline 701 is responsible for determining and tracking the positions of the people three dimensionally. The fine motion detection pipeline 702 detects the finer movements that are below the thresholds of the CFAR (Constant False Alarm Rate) detection block. It begins by applying beam forming to increase the signal-to-noise-ratio (SNR) of the range spectrum in the direction of interest. Then it estimates the phase angle of the range spectrum bins within the range of interest. The phase angle is high-pass filtered in order to see changes in it due to movements. The magnitude of the changes are evaluated in the spike detection block and this signal is combined in the motion detection block with the CFAR detections located within the region of interest. If the system is used to detect immobility or risk of pressure ulcers, in one of the embodiments of the invention output of the motion detection is a reset signal for the timer, which is running whenever the tracking algorithm determines there is a person in the bed. An alarm can be triggered when the timer reaches the predefined safety limit.

One embodiment of the invention relates to a sensor arrangement for observing the presence, location, movement and/or attitude of a person in the monitored area, the sensor arrangement comprising an infrared based sensor, such as a PIR (Passive Infrared)-sensor, and a radar-based sensor. The sensor arrangement further comprises a means for processing the measurement signals of the sensors, such as measuring electronics. The infrared based sensor is configured to measure the monitored area and detect movement of the person in the monitored area. The radar-based sensor is configured to measure the monitored area and detect movement of the person in the monitored area. The sensor arrangement is configured to use the infrared based sensor for observing movement in the monitored area and to enable the radar-based sensor to measure when the infrared based sensor is not able to observe movement in the monitored area and/or the observed movement by the infrared based sensor in the monitored area when it is below a predefined threshold level. The radar-based sensor can be disabled when it's not needed, e.g. when it has not detected any movement or when no accurate measurement results are not needed. Then the monitoring of the area can be continued with the infrared based sensor.

In one embodiment of the invention, the sensor arrangement is configured to disable the radar-based sensor, when the infrared based sensor is able to observe movement in the monitored area and/or the observed movement by the infrared based sensor in the monitored area, when it is above the predefined threshold level.

In one embodiment of the invention, the measurement area of the infrared based sensor is arranged and/or limited so that the infrared based sensor cannot observe movement below a certain height, e.g. at floor level.

In one embodiment of the invention, the sensor arrangement comprises a battery configured to provide energy for the sensor arrangement. In one embodiment of the invention, the sensor arrangement comprises a mains electricity power supply configured to provide energy for the sensor arrangement and/or the battery.

In one embodiment of the invention, the sensor arrangement structure comprises an attachment structure, in which the sensor arrangement can be placed, wherein the attachment structure is fixable on a stand, a wall or ceiling. In one embodiment of the invention, the sensor arrangement is removable from the attachment means without any tools e.g. for charging the battery of the sensor arrangement. The sensor arrangement or attachment structure for the sensor arrangement can be arranged on a stand, a wall, e.g. at the height or higher than the height of 1.5 m from the floor level.

In one embodiment of the invention, the radar-based sensor is configured to determine objects and their azimuth, elevation and/or distance from the sensor based on the measurement signal.

In one embodiment of the invention, the sensor arrangement is configured to analyse the measurement signal by at least filtering the measurement signal in such a way that the phase of the measurement signal is determined in order to observe movement of the person, such as heartbeat and/or breathing.

In one embodiment of the invention, the sensor arrangement is configured to detect falling and/or sitting of the person by the determined elevation of the person, e.g. such that when the elevation of the person is under a certain threshold elevation value, the person can be determined to have fallen.

In one embodiment of the invention, the radar-based sensor is a radar sensor configured to observe the elevation, azimuth, movement and/or distance of objects, e.g. with continuous-wave radar technique, such as a frequency-modulated continuous-wave (FMCW).

In one embodiment of the invention, the sensor arrangement comprises a means to detect the attitude of the sensor, such as an accelerometer, and the sensor is configured to take the attitude of the sensor into account when determining azimuth, elevation and/or distance from the sensor to the person.

One embodiment of the invention relates also to the method for observing the presence, location, movement and/or attitude of a person in a monitored area with a sensor arrangement, the sensor arrangement comprising an infrared based sensor, such as a PIR-sensor, and a radar-based sensor, the sensor arrangement further comprising a means for processing the measurement signals of the sensors, such as measuring electronics. The infrared based sensor measures the monitored area and detects movement of the person in the monitored area. The radar-based sensor measures the monitored area and detects movement of the person in the monitored area. The sensor arrangement uses the infrared based sensor for observing movement in the monitored area and enables the radar-based sensor to measure when the infrared based sensor is not able to observe movement in the monitored area and/or the observed movement by the infrared based sensor in the monitored area is below a predefined threshold level.

One embodiment of the invention relates also to a system for observing the presence, location, movement and/or attitude of one or more objects to be monitored in the area to be monitored. The system comprises at least one sensor arrangement according to the invention, wherein the sensor arrangement or sensor arrangements are fitted in the monitored area, e.g. on a stand, a floor, wall and/or ceiling.

In one embodiment of the invention, the system comprises at least two said sensor arrangements of the invention, and the system is configured to detect and measure the persons in the monitored area based on the measurement signal of at least two sensor arrangements, which can monitor the same area and/or different area.

In one embodiment of the invention, the system is further adapted to send a notification of a fall, if a person is interpreted as having fallen and/or if the vital functions of the monitored person, such as tracked heartbeat and/or breathing of the monitored person, is not within the predefined limits.

In one embodiment of the invention, the notification and/or fall notification is the sending of an alarm or message to a person and/or an organization monitoring the health of the person, e.g. as a message to a phone, as an alarm and/or e.g. to a nurse, to relatives or to an emergency centre.

In one embodiment of the invention, the system is adapted to send information derived from the person onwards, using a wireline or wireless communications means.

In one embodiment of the invention the sensor, sensor arrangement and/or sensor system comprises at least one light source, e.g. a LED light source, wherein the sensor arrangement is configured to activate the light source when the sensor observes a standing person, e.g. at certain times of the day and/or when the light level in the monitored area is low. The sensor arrangement can comprise a means to measure light level in the monitored area.

Usually radar based sensors consume more energy than infrared based sensors. The solution of the embodiment with a sensor arrangement, comprising both of the radar-based sensors and the infrared based sensor is able to lower the energy consumption of the monitoring solution and thus make it possible that the sensor arrangement can be powered e.g. by battery power. In the solution of the invention, the sensor arrangement uses the infrared based sensor for observing movement in the monitored area and enables the radar-based sensor to measure only when it's needed, e.g. when more accurate or detailed measurements are needed. This can be implemented e.g. with the measurement area of the infrared based sensor being arranged and/or limited so that the infrared based sensor cannot observe movement below a certain height, e.g. on the floor level. In this case the infrared based sensor is not able to observe movement in the monitored area and/or the observed movement by the infrared based sensor in the monitored area when it is below a predefined threshold level and more accurate measurements can be carried out with radar-based sensor, for example, for a fallen person. In this situation a radar-based sensor is used and when accurate measurements are not needed the radar-based sensor can be disabled and the area can be monitored with the infrared based sensor.

It is obvious to the person skilled in the prior art that the different embodiments of the invention are not limited solely to the examples described above, and that they may, therefore, be varied within the scope of the claims presented below. The characteristic features possibly presented in the description in conjunction with other characteristic features can also, if necessary, be used separately from each other.

1. A sensor for observing the presence, location, movement and/or attitude of a person in a monitored area, wherein the sensor comprises:
means for processing the measurement signal of the sensor; and
means for communicating measurement results and/or data relating to the measurement results for further processing,
wherein, the sensor is a radar-based sensor configured to detect persons in the monitored area, and to measure and detect movement,
wherein the sensor or a monitoring system connected to the sensor is configured to determine at least one of the following states of the person:
breaks or interruptions with breathing of the monitored person; and/or
immobility of the monitored person, and
wherein the sensor and/or the monitoring system is configured to provide an alarm based on the determined state of the person.

2. The sensor, according to claim 1, wherein the alarm is a local alarm comprising at least one of the following: an audible alarm, headphones or a hearing aid device, a visual alarm, and/or an alarm causing vibrations to the bed, mattress and/or to the monitored person, and/or wherein the local alarm is an alarm on a wearable device, wherein the alarm is vibrating on the wearable device and/or an electric shock is caused by the wearable device.

3. The sensor, according to claim 2, wherein the sensor is configured to recognize breaks or interruptions in the person's breathing, in such a way that if no movement of the person, caused by breathing, is determined over a predefined duration, this is recognized as a break or interruption with breathing.

4. The sensor, according to claim 1, wherein the sensor and/or the system is configured to recognize immobility of the person in such a way that if no movement of the person is determined for a predetermined duration, the person is determined to be immobile.

5. The sensor, according to claim 1, wherein the sensor and/or the system is configured to provide the local alarm until the person is determined to have moved, woken up and/or to have started to breathe again.

6. The sensor, according to claim 1, wherein the sensor and/or the system is configured to provide a remote alarm based on the determined state of the person, by sending a message or alarm to a remote system and/or to a mobile device.

7. The sensor, according to claim 5, wherein the remote alarm is provided, if the person does not respond to the local alarm.

8. The sensor, according to claim 1, wherein the sensor is arranged on a stand, floor, ceiling or wall of a room in a home-environment or a hospital environment.

9. The sensor, according to claim 1, wherein the sensor comprises a first operating mode and a second operating mode,
wherein, in the first operating mode the sensor is configured to track movement of the monitored person, and
wherein in the second operating mode the sensor is configured to measure and/or further analyse measurements relating to a part of the monitored area, in which movement of a person was observed in the first operating mode in order to observe the status of the person.

10. The sensor, according to claim 1, wherein in the second operating mode, the sensor is configured to analyse the measurement signal in such a way that the phase of the measurement signal is determined in order to observe movement of the person.

11. The sensor, according to claim 1, wherein the sensor is a radar sensor configured to observe the elevation, azimuth, movement and/or distance of objects.

12. The sensor, according to claim 1, wherein the sensor comprises a means for detecting the orientation of the sensor, and the sensor is configured to take the detected orientation of the sensor into account when determining measurement results for the monitored person.

13. The sensor, according to claim 1, wherein the sensor is configured to determine sleep quality of the monitored person at least in part, based on monitored breathing frequency and/or the amount of body movement of the monitored person, and/or wherein the determined sleep quality of the monitored person comprises information related to at least one of the following:
how long the person has slept;
how much the person has moved while sleeping;
what kind of sleep phases the monitored person has had while sleeping.

14. A method for observing the presence, location, movement and/or attitude of a person in a monitored area, wherein a sensor is used in the method, which comprises a means for processing the measurement signal of the sensor, and a means for communicating measurement results and/or data relating to the measurement results for further processing, wherein the sensor is a radar-based sensor, the method comprising the steps of:
the sensor or a monitoring system connected to the sensor detecting the persons in the monitored area and measuring and detecting movement;
the sensor determining at least one of the following states of the person: breaks or interruptions with breathing of the monitored person; and
the sensor and/or a monitoring system providing an alarm based on the determined state of the person.

15. The method, according to claim 14, wherein the alarm is a local alarm comprising at least one of the following: an audible alarm, a visual alarm, and/or an alarm causing vibrations to the bed, mattress and/or to the monitored person, and/or wherein the local alarm is an alarm on a wearable device, wherein the alarm is vibrating on the wearable device and/or an electric shock is caused by the wearable device.

16. The method, according to claim 14, wherein the sensor recognizes breaks or interruptions in the person's breathing, in such a way that if no movement of the person caused by breathing is determined over a predefined duration, this is recognized as a break or interruption with breathing.

17. The method, according to claim 14, wherein the sensor and/or the system recognizes immobility of the person in such a way that if no movement of the person is determined for a predetermined duration, the person is determined to be immobile.

18. The method, according to claim 14, wherein the local alarm is provided until the person is moving, waking up and/or starts to breathe again.

19. The method, according to claim 14, wherein the sensor and/or a system provides a remote alarm based on the determined state of the person, by sending a message or alarm to a remote system and/or to a mobile device.

20. The method, according to claim 19, wherein the remote alarm is provided if the person does not respond to the local alarm.

21. The method, according to claim 14, wherein the sensor comprises a first operating mode and a second operating mode,
wherein in the first operating mode the sensor tracks movement of the monitored person, and
wherein in the second operating mode the sensor measures and/or further analyses measurements relating to a part of the monitored area, in which movement of a person was observed in the first operating mode in order to observe the status of the person.

22. The method, according to claim 14, wherein in the second operating mode the sensor analyses the measurement signal in such a way that the phase of the measurement signal is determined in order to observe movement of the person.

23. The method, according to claim 14, wherein the sensor comprises a means for detecting the orientation of the sensor, and the sensor takes the detected orientation of the sensor into account when determining measurement results for the monitored person.

24. The method, according to claim 14, wherein the sensor determines sleep quality of the monitored person at least in part based on monitored breathing frequency and/or the amount of body movement of the monitored person and/or wherein the determined sleep quality of the monitored person comprises information related to at least one of the following:
how long the person has slept;
how much the person has moved while sleeping; and
what kind of sleep phases the monitored person has had while sleeping.

25. A system for observing the presence, location, movement and/or attitude of one or more objects in a monitored area, wherein the system comprises at least one sensor according to claim 1, and
wherein the sensor or sensors are fitted in the monitored area.

26. The system, according to claim 25, wherein the system is configured to provide the local alarm and/or the remote alarm.

27. The system, according to claim 25, wherein the sensor is arranged beside or above a bed so that the measurement area of the sensor covers at least a part of the bed and/or a person lying on the bed, and the sensor is arranged to monitor a person on the bed.
